(19) United States
(12) Patent Application Publication
(10) Pub. No.: US 2026/0159882 A1
(43) Pub. Date: Jun. 11, 2026

(54) DIRECT METHYLATION SINGLE-BASE RESOLUTION SEQUENCING OF ENRICHED UNMETHYLATED DNA

(71) Applicant: GUARDANT HEALTH, INC., Palo Alto, CA (US)

(72) Inventors: Andrew KENNEDY, San Diego, CA (US); James GHADIALI, San Diego, CA (US)

(21) Appl. No.: 19/276,680

(22) Filed: Jul. 22, 2025

Related U.S. Application Data
(60) Provisional application No. 63/675,082, filed on Jul. 24, 2024.

(51) Int. Cl. C12Q 1/6869; C12Q 1/34; C12Q 1/48; C12Q 1/6804; C12Q 1/6806; C12Q 1/6886

(52) U.S. Cl. CPC C12Q 1/6869; C12Q 1/34; C12Q 1/48; C12Q 1/6804; C12Q 1/6806; C12Q 1/6886; G01N 2333/91097; G01N 2333/978

(57) ABSTRACT

Described herein are methods and composition related to detection of unmethylated DNA regions with high sensitivity and low costs compared to current methods. This approach involves tagging CpG in unmethylated DNA with reagents that provides both handle for immobilization/enrichments, and blocking of CpG deamination by deaminases (e.g. APOBEC3A). By leveraging methods involving methyltransferase-based transfer of unnatural substrates to unmodified cytosines (in CpGs only), one can adapt such steps into various workflow sequences involving hyper-and hypo-methylated DNA with flexible deployment of tagging steps as suited for the analyte of interest.

### CROSS-REFERENCE TO RELATED APPLICATION

This application claims the benefit of U.S. Provisional Patent Application No. 63/675,082, filed Jul. 24, 2024, which is incorporated by reference herein in its entirety.

### BACKGROUND

Current hypomethylation interrogation methods, suffer from low specificity and no molecular-level methylation pattern identification (enrichment-based) or high sequencing cost, low sensitivity and reduce somatic detection capability (single-base conversion-based).

As enrichment step does not have perfect specificity for unmethylated molecules and molecule-level methylation patterns have clinical utility in resolving, performing the single-base methylation conversion/sequencing (deamination-based) on the enriched material enhances specificity and utility of enrichment alone. Conversely, conversion on unenriched DNA, will have lower molecular sensitivity and/or higher sequencing costs than enrichment and conversion.

Described herein is a streamlined, high efficiency method to interrogate unmethylated DNA regions with high sensitivity and low costs compared to current methods. This approach involves tagging CpG in unmethylated DNA with reagents that provides both 1) handle for immobilization/enrichments, and 2) blocker of CpG deamination by deaminases (e.g. APOBEC3A). This approach leverages two methods involving methyltransferase-based transfer of unnatural substrates to unmodified cytosines (in CpGs only).

### SUMMARY OF THE INVENTION

Described herein is a method for detecting the methylation profile of nucleic acids in a sample, wherein the method includes: partitioning the nucleic acids based on the presence or absence of 5 hydroxymethylcytosine (5hmC) nucleic acid bases in the nucleic acids, subjecting the nucleic acids to a conversion procedure that selectively converts the base pairing specificity of 5-methylcytosines (5mC) or unmethylated cytosines (C) in the nucleic acids, amplifying the nucleic acids which have been subjected to these steps to generate amplification products, sequencing the amplification products to obtain sequencing data, and analysing the sequencing data to determine whether the cytosine nucleic acid bases of the nucleic acids in the sample are 5hmC, 5mC or C. In other embodiments, the partitioning is performed before the conversion procedure. In other embodiments, the conversion is performed before partitioning. In other embodiments, the partitioning provides at least two subsamples of nucleic acids, wherein a first subsample is enriched for nucleic acids including 5hmC nucleic acid bases and wherein a second subsample is depleted of nucleic acids including 5hmC nucleic acid bases, wherein amplifying, sequencing and/or analyzing are performed on at least the first subsample and/or at least the second subsample. In other embodiments, the partitioning includes modifying the 5hmC nucleic acid base by attaching an isolation tag and partitioning using an agent which binds to the isolation tag. In other embodiments, the method further includes, prior to partitioning and/or the conversion procedure, incubating the nucleic acids with ß-glucosyltransferase and a uridine diphosphoglucose (UDP-Glu) molecule to glycosylate 5hmC nucleic acid bases in the nucleic acid molecule with a glucose molecule, optionally wherein the UDP-Glu is a modified UDP-Glu and the glycosylation of 5hmC is with a modified glucose molecule. In other embodiments, partitioning includes generation of one or more of: a primary, secondary, or untagged hyper-partition, a primary, secondary, or untagged hypo-partition, and a primary, secondary, or untagged other partition. In other embodiments, the partitioning includes generation of a hyper-partition, a hypo-partition, and/or other partition. In other embodiments, the partitioning includes generation of a primary tagged hyper-partition, a hypo-partition, and/or other partition. In other embodiments, the partitioning includes generation of a secondary tagged a hyper-partition, a hypo-partition, and/or other partition. In other embodiments, the partitioning includes generation of a hyper-partition, and a primary tagged hypo-partition, and a secondary tagged a hypo-partition. In other embodiments, the partitioning includes generation of a primary tagged hypo-partition, a hyper-partition, and a secondary tagged hypo-partition. In other embodiments, the partitioning includes generation of a primary tagged hypo-partition, a secondary tagged hypo-partition, and a hyper-partition. In other embodiments, the partitioning includes generation of a secondary tagged hypo-partition,, a hypo-partition, and a hyper-In other embodiments, the partitioning includes generation of a primary tagged hypo-partition, and a secondary tagged hypo-partition, and a hyper-partition. In other embodiments, the modified UDP-Glu includes an azide linker and/or a thiol linker. In other embodiments, the modified UDP-Glu includes an isolation tag which is used in the partitioning step. In other embodiments, the isolation tag is biotin or a histidine tag. In other embodiments, the partitioning includes one or more of: reacting the modified glucose with an isolation tag, and binding the glycosylated 5hmC with J binding protein 1 (JBP1). In other embodiments, the isolation tag is an isolation tag including biotin. In other embodiments, the partitioning includes exposing the nucleic acids to a binding agent which selectively binds 5hmC. In other embodiments, the binding agent is an anti-5hmC antibody, or an antigen-binding fragment thereof. In other embodiments, the conversion procedure selectively converts the base pairing specificity of 5-methylcytosines (5mC) in the nucleic acids. In other embodiments, the conversion procedure includes Tet-assisted conversion of nucleic acids with a substituted borane reducing agent, wherein 5hmC nucleic acid bases are protected from conversion, optionally through glucosylation. In other embodiments, the substituted borane reducing agent is 2-picoline borane, borane pyridine, tert-butylamine borane, ammonia borane or pyridine borane. In other embodiments, the conversion procedure includes: reacting the nucleic acids with a variant methyltransferase having carboxymethyltransferase activity in the presence of carboxy-S-adenosyl-L-methionine (CxSAM) substrate, thereby labelling any unmethylated C and rendering it resistant to deaminase action, wherein 5hmC nucleic acid bases are protected from conversion through glucosylation, and contacting the nucleic acids of step (i) with a deaminase enzyme which is APOBEC3A. In other embodiments, the conversion procedure includes unmethylated cytosine (C), 5-methylcytosine (5 mC) and 5-hydroxymethylcytosine (5hmC) in nucleic acids in the sample. In other embodiments, the conversion procedure, includes: reacting a polynucleotide containing C, 5mC, and/or 5hmC with a variant methyltransferase having carboxymethyltransferase activity in the presence of carboxy-S-adenosyl-L-methionine (CxSAM) substrate, thereby labeling any unmodified C in said nucleic acids and rendering it resistant to deaminase action, wherein said 5hmC is also optionally glucosylated, contacting the nucleic acids of step (a) with a deaminase which deaminates 5mC and/or 5hmC, with minimal damage to said target nucleic acids present in said sample, analyzing said nucleic acids sample, to identify each of unmodified C, 5mC, and 5hmC present in said nucleic acids. In other embodiments, the nucleic acids in the sample are fragmented or sheared prior to partitioning and/or the conversion method.

In other embodiments, sequencing includes use of sequence adapters containing modified cytosine bases resistant to deamination. In other embodiments, the nucleic acids in the sample are amplified prior to sequencing. In other embodiments, the variant methyltransferase having carboxymethylase activity is a recombinant M. Mpel N374K and said In other embodiments, the deaminase enzyme is APOBEC3A. In other embodiments, the modified cytosine base is 5pyC.

In other embodiments, the DNA is genomic DNA. In other embodiments, the NA is cell-free DNA (cfDNA). In other embodiments, the method includes inclusion of methylated control nucleic acids s. In other embodiments, the nucleic acids are obtained from cancer cells. In other embodiments, comparison with results obtained using bisulfite dependent 5mC+5hmC localization and ACE-seq 5hmC localization. In other embodiments, the variant methyltransferase having carboxymethylase activity is a recombinant M. Mpel N374K. In other embodiments, the conversion procedure selectively converts the base pairing specificity of unmethylated cytosines (C) in the nucleic acids. In other embodiments, the conversion procedure is bisulfite conversion. In other embodiments, analyzing the sequencing data further includes identifying the presence or absence of genetic variants. In other embodiments, the genetic variants are selected from a single nucleotide variant (SNV), an insertion or deletion (indel), and a copy number variation. In other embodiments, the 5hmC methylation status of the nucleic acids in the sample is determined by analyzing the base coverage of cytosines in a reference sequence. In other embodiments, the method includes enriching the nucleic acids by capturing a target region set from the sample, wherein the capture step is before, after or in between the partitioning step and the conversion procedure step, or between other steps. In other embodiments, the nucleic acids comprise DNA, optionally cell-free DNA (cfDNA) obtained from a subject, optionally wherein the subject is a patient having or suspected of having cancer. In other embodiments, the method includes using the detection of the methylation status in the nucleic acids to determine or predict the presence or absence of nucleic acids produced by a cancer cell or tumor, to determine the probability that a subject has a tumor or cancer, or to characterize a cancer or tumor of the subject. In other embodiments, the target region set further includes one or more epigenetic target region sets. In other embodiments, the target region set further includes one or more sequence-variable target region sets. In other embodiments, the method includes amplifying the enriched nucleic acids of the first subsample prior to combining the enriched nucleic acids of the first subsample and the nucleic acids of the second subsample. In other embodiments, the method includes amplifying includes one or more of polymerase chain reaction, linear amplification, rolling circle amplification, ligase chain reaction, strand displacement amplification, nucleic acid sequence-based amplification, and self-sustained sequence-based replication. In other embodiments, the amplification includes thermocycled amplification. In other embodiments, the amplification includes isothermal amplification. In other embodiments, the nucleic acids of the first subsample and the nucleic acids of the second subsample are differentially tagged. In other embodiments, the nucleic acids comprise barcodes. In other embodiments, the nucleic acids further comprise adapters in which at least one cytosine is a modification resistant cytosine, optionally wherein each cytosine in the adapters is a modification resistant cytosine. In other embodiments, the method includes ligating adapters to the nucleic acids, wherein at least one cytosine in the adapters is a modification resistant cytosine); further optionally wherein each cytosine in the adapters is a modification resistant cytosine. In other embodiments, the modification resistant cytosine is a deaminase resistant cytosine. In other embodiments, the deaminase resistant cytosine is 5-propynyIC (5 pyC), 5-pyrrolo-dC (5pyrC), 5-hydroxymethylcytosine (5hmC), glucosylated 5-hydroxymethylcytosine (5ghmC), cytosine 5-methylenesulfonate (CMS), or N4-modified cytosine. In other embodiments, the adapters comprise barcodes. In other embodiments, the method includes ligating adapters including barcodes to the amplification products prior to the sequencing. In other embodiments, the method includes ligating adapters including barcodes to the nucleic acids prior to the amplifying. In other embodiments, the sequencing includes next generation sequencing. In other embodiments, sequencing includes long-read sequencing. In other embodiments, the sequencing includes nanopore sequencing. In other embodiments, sequencing the nucleic acids of the amplification products includes generating a plurality of sequencing reads; and the method further includes mapping the plurality of sequence reads to one or more reference sequences to generate mapped sequence reads, and processing the mapped sequence reads corresponding to a sequence-variable target region set and to an epigenetic target region set. In other embodiments, the nucleic acids comprise cell-free DNA, optionally wherein the cell-free DNA is in an amount between 1 ng and 500 ng. In other embodiments, the nucleic acids comprise DNA from a blood sample and/or a tissue sample. In other embodiments, the blood sample is a whole blood sample, a plasma sample, a buffy coat sample, a leukapheresis sample, or a PBMC sample. In other embodiments, the nucleic acids and/or the sample is from a subject. In other embodiments, the subject is an animal. In other embodiments, the subject is a human. In other embodiments, the method includes r localizing 5 mC modifications in the genome which accurately profiles the methylome is provided. An exemplary method entails resolving unmethylated cytosine (C), 5-methylcytosine(5 mC) and 5-hydroxymethylcytosine (5hmC) in a nucleic acids sample by a) reacting a nucleic acids optionally containing C, 5mC, and/or 5hmC with a variant methyltransferase in the presence of carboxy-S-adenosyl-L-methionine (CxSAM) substrate, thereby labeling any unmodified C in said nucleic acids and rendering it resistant to deaminase action; b) contacting the nucleic acids above with a deaminase which deaminates 5mC and/or 5hmC, with minimal damage to said target nucleic acids present in said sample; and c) sequencing the deaminated nucleic acids sample, thereby identifying each of unmodified C, 5mC, and 5hmC present in said nucleic acids. In certain embodiments, the nucleic acids s in the sample are fragmented or sheared prior to step a), and sequence adapters containing modified cytosines resistant to deamination, such as 5pyC, are operably linked to said sheared or fragmented nucleic acids. In other embodiments, the sample of step b) is amplified prior to the sequencing of step c). In preferred embodiments of the invention, the variant methyltransferase is a recombinant M.Mpel N374K and the deaminase enzyme is APOBEC3A. The nucleic acids sample can be from any source and in certain aspects, includes genomic DNA, cancer cell DNA, cell free DNA or DNA in maternal circulation. The method can also optionally include methylated control nucleic acids s. In other embodiments, the method can further comprise the step of comparing results obtained with those obtained using bisulfite dependent 5mC localization and ACE-seq 5hmC localization.

In a further embodiment of the invention, a kit for practicing the methods described above are provided. In one aspect, the kit including a variant M.Mpel methyltransferase. In yet another aspect, the kit further includes a cytosine deaminase enzyme which can be the deaminase enzyme, APOBEC3A. The kit of the invention can further comprise reagents and materials for cleaving or shearing DNA. In yet another approach the kit can further comprise including reagents for amplification of DNA.

In an optional embodiment of this method, the DNA can be end-repaired, A-tailed, and forkhead full-length Illumina adapters can be installed with indices unique to each individual sample type (e.g. Illumina TruSeq DNA Library Prep LT or HT). While all workflow and reagents will remain the same for standard Illumina TruSeq library prep, custom solid-phase synthesized adapters, replacing all Cs with deamination-resistant cytosine analogs, such as 5pyCs, will be used in place of standard Illumina adapters. Although the workflow described can be used for Illumina libraries, adapters should be utilized to pre-adapt any sequencing adapters before A3A or bisulfite based sequencing approaches. In preferred embodiments, given the preference of the CxMTase for introducing 5cxmC at unmodified CpGs when the opposite strand contains a 5mCpG, this idealized substrate can be generated by a single copy step of the template strand using Klenow (exo-) polymerase or another displacing polymerases, along with 5mdCTP in lieu of dCTP in the dNTP mix.

In the standard embodiment of this method (without preadapted DNA), post A3A treated DNA is then prepared with any post-bisulfite adapter ligation strategy, optionally, locus-specific analysis can be performed with direct amplification of either post A3A treated DNA or library prepped DNA at loci of choice using bisulfite primers. Reads can be sequenced on any sequencing platform and can be additionally aligned using any bisulfite-sequencing based bioinformatic strategy.

### BRIEF DESCRIPTION OF THE DRAWINGS

FIG. 1: DM-seq involves use of two enzymes: MTase-based tagging of DNA and Cytosine deaminase reaction (e.g. APOBEC3A)-deaminates methylated cytosines in CpG context and all cytosines in CpH contest. This results in observed TpG=methylated CpG, observed CpG=unmethylated CpG). An important feature is that deamination reaction occurs after original MTase tagging and before amplification. Different library-preps (.e.g ssDNA prep) can also be used and performed after deamination. One of ordinary skill readily appreciates that the order of steps in exemplary illustrative protocol above may be altered. Here, one workflow sequence involves hyper-partitioning, primary hypo-tagging, secondary hypo-tagging and partitioning as depicted.

FIG. 2: An additional workflow sequence includes primary hypo-tagging, hyper-partitioning, secondary hypo-tagging and partitioning. In some instances, this may be used in a methyl binding domain (MBD) using his tag (non-biotin).

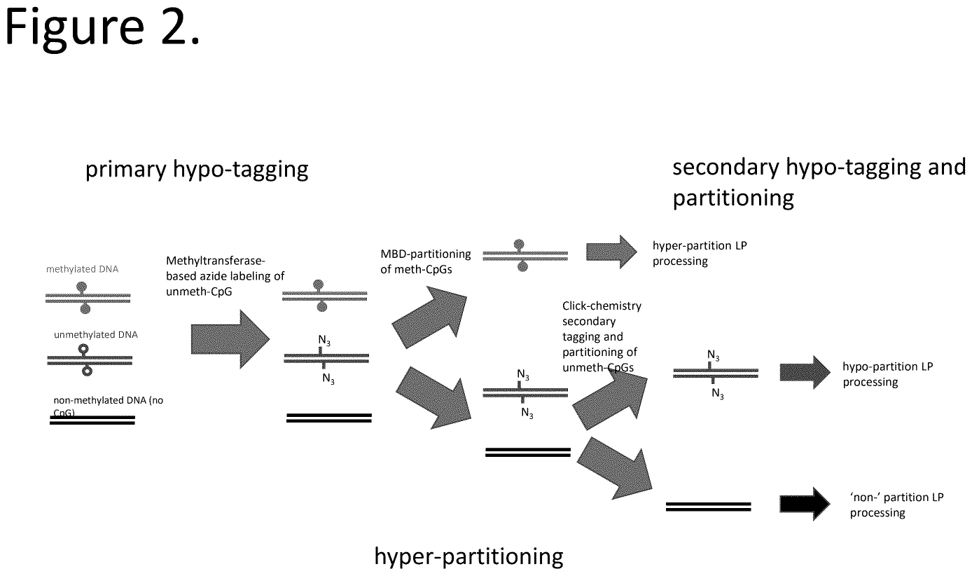

FIG. 3: An additional workflow sequence includes primary hypo-tagging, secondary hypo-tagging and partitioning, hyper-partitioning.

FIG. 4: An additional workflow sequence includes primary+secondary hypo-tagging, hypo-partitioning, hyper-partitioning.

FIG. 5: An additional workflow sequence includes primary+secondary hypo-tagging, hypo-partitioning, hyper-partitioning. Here, secondary hypo tagging with immobilization affinity reagent, i.e. biotin/streptavidin, must be orthogonal to immobilization affinity pair used in hyper partitioning (i.e. MBD-Fc/protein A, G)

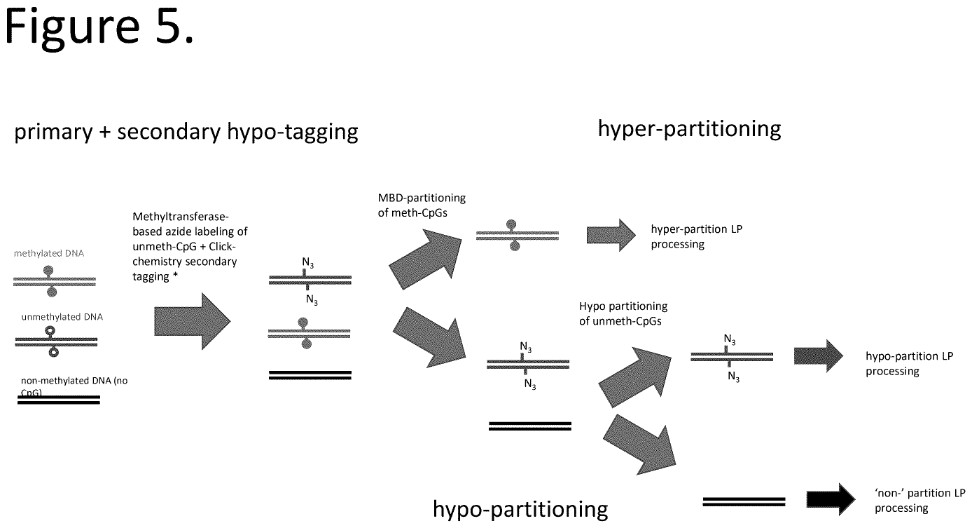

### DETAILED DESCRIPTION

Direct methylation sequencing (DM-Seq) is a bisulfite-free method for profiling 5mC at single-base resolution using as little as nanogram quantities of DNA. DM-Seq employs two key DNA-modifying enzymes: (1) a neomorphic DNA methyltransferase (MTase) and a (2) DNA deaminase capable of precise discrimination between cytosine modification states. Coupling these activities with deaminase-resistant adapters enables accurate detection of only 5mC via a C-to-T transition in sequencing. It has been reported that this approach remedies a PCR-related underdetection bias with conventional hybrid enzymatic-chemical TET-assisted pyridine borane sequencing approach and unlike bisulfite sequencing, unmasks CpGs in clinical tumor samples by not confounding 5mC with 5-hydroxymethylcytosine. See Wang, et al. (2024), incorporated by reference herein.

DM-Seq relies on efficient transfer of the protecting group to unmodified CpGs and complete protection of the newly generated modified base from A3A-mediated deamination. To prevent deamination, 5cxmC, has features of both size and negative charge that can be disfavored by A3A and DM-Seq exploits this property through use of CxMTase: CxSAM enzyme: substrate pair for further development. One can tag CpG in unmethylated DNA with reagents to provide both 1) handle for immobilization/enrichments, and 2) blocker of CpG deamination by deaminases (e.g. APOBEC3A). This includes for example click-chemistry reaction with immobilization affinity pair (DBCO-biotin) and end-repair, A-tailing (optional), adapter ligation (cytosine in adapter protected from deaminase conversion). This approach leverages two methods involving methyltransferase-based transfer of unnatural substrates to unmodified cytosines (in CpGs only).

Illumina TruSeq Y-shaped adapters, with all Cs replaced with 5pyC bases, are not impacted by the presence of 5pyC bases which is then utilized in DM-Seq workflo. First 5 pyC adapters are ligated to DNA and copied to create a strand exclusively containing 5mCs in place of C. The DNA is then protected by the CxMTase (acting on unmodified CpGs) and glucosylation by βGT (for 5hmCs). Subsequent deamination by A3A is performed before PCR amplification and sequencing.

### Ligation of Adapters

Double-stranded nucleic acids e.g., DNA molecules in a sample, and single stranded nucleic acid molecules converted to double stranded molecules, can be linked to adapters at either one end or both ends. In the methods of the disclosure, adapters can be ligated to sample nucleic acids prior to the partitioning and/or conversion steps. In some embodiments, adapters may be ligated to the sample nucleic acids after the partitioning and conversion steps, but before the step of amplifying the nucleic acids which have been subjected to partitioning and conversion steps.

In some embodiments, the DNA is made ligatable, e.g., by extending the end overhangs of the DNA molecules, and adding adenosine residues to the 3′ ends of fragments and phosphorylating the 5′ end of each DNA fragment. Typically, double stranded molecules are blunt ended by treatment with a polymerase with a 5′-3′ polymerase and a 3′-5′ exonuclease (or proof reading function), in the presence of all four standard nucleotides. Klenow large fragment and T4 polymerase are examples of suitable polymerase.

The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the sample nucleic acid molecules and the adapters have blunt ends. In sticky-end ligation, typically, the sample nucleic acid molecules bear an “A” overhang and the adapters bear a “T” overhang.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample nucleic acid molecule. In some instances, two adapters can be ligated to a single sample nucleic acid molecule, with one adapter ligated to each end of the sample nucleic acid molecule.

Adapters can include nucleic acid primer binding sites to permit amplification of a sample nucleic acid molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given nucleic acid molecule. Adapters of the same or different sequence can be linked to the respective ends of a sample nucleic acid molecule. In some embodiments, adapters of the same or different sequence are linked to the respective ends of the nucleic acid molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a nucleic acid molecule to be analyzed.

Other exemplary adapters include T-tailed, C-tailed or hairpin shaped adapters. For example, a hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times.

In some embodiments, the nucleic acids further comprise adapters in which at least one cytosine is a modification resistant cytosine, optionally wherein each cytosine in the adapters is a modification resistant cytosine. In some embodiments, methods further comprise further comprising ligating adapters to the nucleic acids, wherein at least one cytosine in the adapters is a modification resistant cytosine, optionally wherein the ligating occurs before step (c) and/or after step (a); further optionally wherein each cytosine in the adapters is a modification resistant cytosine. The adapters may comprise barcodes, e.g., according to any of the embodiments relating to barcodes described elsewhere herein. In some embodiments, the adapters can include at least 1, 2, 3, 4, 5, 6, 7, 8, 9, 10, 11, 12, 13, 14, 15, 16, 17, 18, 19, or 20 modified nucleotides, such as modified cytosine nucleotides, that are resistant to modification, e.g., conversion. In some embodiments, the modified nucleotides are resistant to modification by a deaminase. In some embodiments, the modified nucleotides comprise a conversion resistant modified cytosine, such as 5-propynyIC (5pyC), 5-pyrrolo-dC (5pyrC), 5-hydroxymethylcytosine (5 hmC) along with modified variants thereof, glucosylated5-hydroxymethylcytosine (5ghmC), cytosine 5-methylenesulfonate (CMS), bulky 5-position adducts, or N4-modified cytosine. In some embodiments, the conversion resistant modified cytosine is 5pyC, 5pyrC, 5ghmC, or CMS. In some embodiments, the conversion resistant modified cytosine can protect cytosine from being converted by a deaminase, such as a cytidine deaminase, which converts a cytosine to uracil. In some embodiments, each cytosine of an adapter is a conversion resistant modified cytosine, such as any one or more of the foregoing examples. For exemplary descriptions of modified nucleotides and their use in adaptors, see WO2023/288222 and U.S. Pat. No. 10,260,088.

The adapters used in the methods of the present disclosure may comprise one or more known nucleosides wherein the base has a known methylation status, such as 5mC nucleic acid bases. When using adapters comprise 5mC, the adapters can be ligated to the sample nucleic acid molecules prior to the conversion procedure. Analyzing the sequence data corresponding to these known 5mC nucleic acid bases allows for the efficiency of the conversion procedure to be measured, which can be used as a quality control measure for the conversion procedure. In instances where two adapters are ligated to a sample nucleic acid (one at each end), either or both of the adapters may comprise one or more nucleosides with a known methylation status. Typically the primer binding site(s), sequencing primer binding site(s), sample index(es) and/or molecular barcode(s), if present, do not comprise the nucleosides with a methylation status that change base pairing specificity as a result of the conversion procedure.

Preferably adapters (e.g., Y-shaped adapters) are ligated to the sample nucleic acids prior to the conversion and partitioning steps.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some such methods, first adapters are added to the 5′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter includes an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the single-stranded DNA library preparation is performed in a one-step combined phosphorylation/ligation reaction, e.g., as described in Troll et al., BMC Genomics, 20:1023 (2019), available at doi.org/10.1186/s 12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

### Molecular Tagging

In some embodiments, the nucleic acid molecules of the sample may be tagged with sample indexes, partition tags and/or molecular barcodes (referred to generally as “tags”). Tags can form part of an adapter.

Tags can be molecules, such as nucleic acids, containing information that indicates a feature of the molecule with which the tag is associated. For example, molecules can bear a sample tag or sample index (which distinguishes molecules in one sample from those in a different sample), a partition tag (which distinguishes molecules in one partition from those in a different partition) and/or a molecular tag/molecular barcode/barcode (which distinguishes different molecules from one another (in both unique and non-unique tagging scenarios). In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member. For example, barcodes can be used to allow the origin of the DNA (e.g., the subject, biological sample (e.g., samples collected at various time points), enriched DNA sample (e.g., enriched DNA comprising an epigenetic target region set or enriched DNA comprising a sequence-variable target region set), partition, or similar) to be identified, e.g., following pooling of a plurality of samples for parallel sequencing.

In the methods of the disclosure, partitioning results in the generation of multiple subsamples (i.e. partitions) based on the presence or absence of 5hmC nucleic acid bases in the sample nucleic acids. Tags can be used to label the nucleic acids in each partition so as to correlate the tag (or tags) with a specific partition. For example, if multiple subsamples are carried forward after the partitioning step, tags can be used to label each of the subsamples such that the corresponding sequence data deriving from each subsample can be identified. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLoS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g., by blunt-end ligation or sticky-end ligation), or overlap extension polymerase chain reaction (PCR), among other methods. Such adapters are ultimately joined to the target nucleic acid molecule. In other embodiments, one or more rounds of amplification cycles (e.g., PCR amplification) may be applied to introduce sample indexes to a nucleic acid using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after the conversion procedure. In some embodiments, molecular barcodes and/or sample indexes are introduced prior to and/or after the partitioning step. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based sequence capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample nucleic acids. In some embodiments, tags are predetermined or random or semi-random sequences. In some embodiments, the tag(s) may together be less than about 500, 200, 100, 50, 20, 10, 9, 8, 7, 6, 5, 4, 3, 2, or 1 nucleotide in length. Typically, tags are about 5 to 20 or 6 to 15 nucleotides in length. The tags may be linked to sample nucleic acids randomly or non-randomly.

In some embodiments, each sample is uniquely tagged with a sample index or a combination of sample indexes. In some examples, when multiple subsamples (i.e. partitions) are subsequently processed after the partitioning step, each partition can be uniquely tagged with a partition tag or a combination of partition tags. In some embodiments, each nucleic acid molecule of a sample or subsample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual nucleic acid molecules such that the combination of the molecular barcode and the sequence of the sample nucleic acid that it is attached to creates a unique sequence that may be individually tracked. Detection of non-unique molecular barcodes in combination with endogenous sequence information typically allows for the assignment of a unique identity to a particular molecule. Endogenous sequence information includes the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample. In some embodiments, beginning region comprises the first 1, first 2, the first 5, the first 10, the first 15, the first 20, the first 25, the first 30 or at least the first 30 base positions at the 5′end of the sequencing read that align to the reference sequence. In some embodiments, the end region comprises the last 1, last 2,the last 5, the last 10, the last 15, the last 20, the last 25, the last 30 or at least the last 30 base positions at the 3′end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers.

In some embodiments, the assignment of unique or non-unique molecular barcodes in reactions is performed using methods and systems described in, for example, U.S. Patent Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths). The addition of tags (e.g., sample indexes, partition tags and/or molecular barcodes) to nucleic acids can be done through amplification, wherein the tags are comprised in primers used for amplification.

In some embodiments, the nucleic acids are ligated to adapters comprising molecular barcodes. These molecular barcodes (optionally in combination with endogenous sequence information) can then be used when analyzing the sequencing data to group sequence reads deriving from the same parent nucleic acids (i.e. those nucleic acids prior to any amplification). The grouped sequence reads can then be analyzed, for example, to determine a consensus sequence for parent nucleic acids. The consensus sequence will include any converted bases and thus can be used to determine the methylation status of the parent nucleic acid. Similarly, the abundance of consensus sequences from a subsample at C positions in a reference can be used to determine the 5hmC status of the parent nucleic acids. For instance, when the base coverage of a specific C position in a reference sequence is higher than other C positions in a subsample which has been enriched for 5hmC, that specific C position on that parent nucleic acid can be identified as comprising a 5hmC modification at that C position.

### Conversion Procedure

The conversion procedures which are used in the methods of the disclosure can either convert: (i) the base pairing specificity of 5mC (e.g. Tet-assisted conversion with a substituted borane reducing agent); or (ii) the base pairing specificity of unmethylated cytosines (e.g., bisulfite conversion). Preferably the methods of the disclosure employ conversion procedures which convert the base pairing specificity of 5mC because such methods allow for increased sensitivity when using the sequencing data to also detect genetic variants.

In conversion procedures wherein the base pairing specificity of unmethylated cytosines is converted, it is difficult to identify the presence or absence of somatic mutations of cytosines in the sample nucleic acids. In contrast, when conversion procedures which convert the base pairing specificity of 5mC are used, unmethylated cytosines are retained, thus allowing C>T/G>A somatic mutations to be detected with high confidence. Moreover, conversion procedures which convert the base pairing specificity of 5mC (such as TAPS β and DM-Seq) are generally not as destructive as conversion procedures which convert the base pairing specificity of unmethylated cytosines (e.g., bisulfite sequence), and thus the fragmentation pattern of the sample nucleic acids is retained. This can be advantageous, e.g., in the analysis of cfDNA. Accordingly, the use of conversion procedures which convert the base pairing specificity of 5mC additionally allows for both sensitive mutation detection and the analysis of the sample nucleic acid fragmentation pattern.

There are various methods of detecting and/or identifying methylated cytosines that rely on a conversion procedure that changes the base-pairing specificity of a cytosine, based on its methylation status. These changes of base-pairing specificity can then be detected, and thus the methylation status of the cytosine inferred, by sequencing.

The methods of the present disclosure involve subjecting the nucleic acids to a conversion procedure that selectively converts the base pairing specificity of 5-methylcytosines (5mC) or unmethylated cytosines (C).

Procedures that selectively convert the base pairing specificity of 5mC refer to methods which convert the base pairing specificity of 5mC but not C. Such procedures can include methods which involve conditions which would also result in the conversion of the base pairing specific of unprotected 5hmC (e.g., TAPS), provided that, in the methods of the disclosure, any 5hmC is protected (e.g., by glucosylation) from conversion.

Procedures that selectively convert the base pairing specificity of C refer to methods which convert the base pairing specificity of C but not 5mC. Such procedures can include methods which involve conditions which would also result in the conversion of the base pairing specific of unprotected 5hmC (e.g., oxidative bisulfite sequencing), provided that, in the methods of the disclosure, any 5hmC is protected from conversion.

In some embodiments, the conversion procedure used in the methods of the disclosure is one that changes the base pairing specificity of 5mC, but does not change the base pairing specificity of unmethylated cytosines. Advantages of methods that do not convert the base-pairing specificity of unmethylated cytosines include reduced loss of sequence complexity, higher sequencing efficiency and reduced alignment losses. Additionally, methods such as TAPS, TAPS β, and DM-Seq may in some cases be preferred over methods such as bisulfite sequencing because they are less destructive (especially important for low yield samples such as cfDNA) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized regions of interest, these non-random (localized) conversion can appear as false negatives (non-methylated regions). Random non-conversion methods can maximally affect a low percent of bases within a region, and thus the specificity of methylation change detection can be maximized (reduce false positives) by placing a threshold on the percentage of bases within a region that are methylated/non-methylated. Hence, in some embodiments, a conversion procedure that does not involve denaturation is preferred.

In other embodiments, the conversion procedure used in the methods of the disclosure is one that changes the base pairing specificity of an unmethylated cytosine, but does not change the base pairing specificity of 5mC. Such methods include, for example, bisulfite sequencing and EM-seq.

In some embodiments, the conversion procedure converts the base pairing specificity 5mC. In some embodiments, the conversion procedure which converts the base pairing specificity of 5mC comprises protection of 5hmC (e.g., using β-glucosyltransferase (GT) or 5-hydroxymethylcytosine carbamoyltransferase) combined with Tet-assisted conversion with a substituted borane reducing agent, e.g., 2-picoline borane, borane pyridine, tert-butylamine borane, ammonia borane or pyridine borane. In this method, 5hmC can be protected from conversion, for example through glucosylation using β-glucosyltransferase (BGT), forming 5-glucosylhydroxymethylcytosine (5ghmC), or through carbamoylation using 5-hydroxymethylcytosine carbamoyltransferase, forming 5cmC. A method of protecting 5hmC from conversion, for example through glucosylation using β-glucosyltransferase (BGT), forming 5-glucosylhydroxymethylcytosine (5ghmC), is described in Yu et al., Cell 2012; 149:1368-80. Alternatively, a carbamoyltransferase enzyme, such as 5-hydroxymethylcytosine carbamoyltransferase as described in Yang et al., Bio-protocol, 2023; 12(17): e4496, can be used to protect hmC (by converting hmC to 5-carbamoyloxymethylcytosine (5cmC)), then a TET protein, such as mTet1 or a TET2 comprising a T1372S mutation, can be used to convert mC to caC, and then bisulfite treatment can be used to convert C and caC to U while 5cmC remains unaffected. In this method, 5hmC can be protected from conversion, for example through glucosylation using β-glucosyltransferase (BGT), forming 5-glucosylhydroxymethylcytosine (5ghmC). Treatment with a TET protein, such as mTet1 or a TET2 comprising a T1372S mutation, then converts 5mC to 5caC but does not convert C, 5ghmC, or 5cmC. 5 caC is then converted to DHU by treatment with pic-borane or another substituted borane reducing agent such as borane pyridine, tert-butylamine borane, or ammonia borane, also without affecting 5ghmC, 5cmC, or unmethylated C. Sequencing of the converted DNA identifies positions that are read as cytosine as being either 5hmC or unmethylated C positions. Meanwhile, positions that are read as T are identified as being T or 5mC. T and 5mC can be distinguished through alignment to a reference sequence. When the corresponding position in a reference sequence is T, the nucleoside on the sample nucleic acid is identified as a T. When the corresponding position in a reference sequence is C, the nucleoside on the sample nucleic acid is identified as a 5mC. For an exemplary description of this type of conversion, see, e.g., Liu et al., Nature Biotechnology 2019; 37:424-429. 5- hydroxymethylcytosine carbamoyltransferase is described in Yang et al., Bio-protocol, 2023; 12(17): e4496. Performing such conversion methods (e.g., TAPS β conversion) on a sample as described herein thus facilitates distinguishing positions containing unmethylated C or 5hmC on the one hand from positions containing 5mC using the sequence reads obtained. The unmethylated C can then be distinguished from the 5hmC by analyzing the sequence data and using the base coverage analysis of subsamples from the partitioning step, wherein higher base coverage of cytosines in the subsample enriched for 5hmC would indicate that those cytosines were 5hmC in the sample nucleic acids corresponding to those sequence reads.

In some embodiments, the conversion procedure which converts the base pairing specificity of 5mC comprises reacting the nucleic acids with a variant methyltransferase having carboxymethyltransferase activity in the presence of carboxy-S-adenosyl-L-methionine (CxSAM) substrate, thereby labelling any unmethylated C and rendering it resistant to deaminase action. When this method is used in the context of the present disclosure, the 5hmC nucleic acid bases are also protected from deaminase action, e.g., through glucosylation such as by BGT. In some embodiments, BGT and CxMTase reactions occur simultaneously. The nucleic acids can then be contacted with a deaminase enzyme (e.g., APOBEC3A) which deaminates 5mC to uracil. Sequencing of the converted DNA identifies positions that are read as cytosine as being either 5hmC or unmethylated C positions. Meanwhile, positions that are read as T are identified as being T or 5mC. T and 5mC can be distinguished through alignment to a reference sequence. When the corresponding position in a reference sequence is T, the nucleoside on the sample nucleic acid is identified as a T. When the corresponding position in a reference sequence is C, the nucleoside on the sample nucleic acid is identified as a 5mC. In some embodiments, the variant methyltransferase having carboxymethylase activity is a recombinant M. Mpel N374K, for example. In some embodiments, the deaminase enzyme is APOBEC3A. For an exemplary description of this type of conversion, known as DM-seq, see WO2021/236778.

In some embodiments, the conversion procedure converts the base pairing specificity of unmethylated cytosines. In some embodiments, the conversion procedure which converts unmethylated cytosines comprises bisulfite conversion. Treatment with bisulfite converts unmethylated cytosine to uracil whereas 5mC and 5hmC are not converted. Thus, where bisulfite conversion is used, the converted nucleobases are inferred as comprising unmethylated cytosine. The unconverted nucleobases are inferred as comprising 5mC and/or 5hmC. Sequencing of bisulfite-treated DNA identifies positions that are read as cytosine as being 5mC or 5hmC. Meanwhile, positions that are read as T are identified as being T or unmethylated cytosine. Thus, performing bisulfite conversion as described herein thus facilitates identifying positions containing 5mC or 5hmC versus positions containing unmethylated C. The 5mC can then be distinguished from the 5hmC by analyzing the sequence data and using the base coverage analysis of subsamples from the partitioning step, wherein higher base coverage of cytosines in the subsample enriched for 5hmC would indicate that those cytosines were 5hmC in the sample nucleic acids corresponding to those sequence reads. For an exemplary description of bisulfite conversion, see, e.g., Moss et al., Nat Commun. 2018; 9:5068.

In some embodiments, the conversion procedure converts unmethylated cytosines and comprises a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the conversion procedure which converts unmethylated cytosines comprises enzymatic conversion of the first nucleobase using MsddA.

In some embodiments, the conversion procedure is an enzymatic conversion procedure which converts the base pairing specificity of modified nucleosides (e.g., DM-seq conversion comprising adding a protective group (such as a carboxymethyl group) to unmodified cytosines, and deaminating 5mC, such as using an APOBEC enzyme) or an enzymatic conversion procedure which converts the base pairing specificity of unmodified nucleosides (such as SEM-seq).

In some embodiments, the conversion procedure used in the methods of the disclosure is one that changes the base pairing specificity of a modified nucleoside (e.g., methylated cytosine), but does not change the base pairing specificity of the corresponding unmodified nucleoside (e.g., cytosine) or does not change the base pairing specificity of any un-modified nucleoside (e.g., cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as DM-seq may in some cases be preferred over methods such as bisulfite sequencing and EM-seq because they are less destructive (especially important for low yield samples such as cfDNA) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized region of interest, these non-random (localized) conversion can appear as false negatives (non-methylated regions). Random non-conversion methods can maximally affect a low percent of bases within a region, and thus the specificity of methylation change detection can be maximized (reduce false positives) by placing a threshold on % of bases within a region that are methylated/non-methylated. Hence, in some cases, a conversion procedure that does not involve denaturation is preferred.

In other cases, the conversion procedure used in the methods of the disclosure is one that changes the base pairing specificity of an unmodified nucleoside (e.g., cytosine), but does not change the base pairing specificity of the corresponding modified nucleoside (e.g., methylated cytosine).

The skilled person can select a suitable method according to their needs, including which nucleoside modifications are to be detected and/or identified.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5 mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36:1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M. SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.Mpel). In a particular embodiment, the methyltransferase enzyme is a variant of M.Mpel, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M. Mpel having an N374R substitution or an N374K substitution. The methyltransferase can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using BGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (BGT), forming (5-glucosylhydroxymethylcytosine) 5ghmC, or through carbamoylation using 5- hydroxymethylcytosine carbamoyltransferase, forming 5cmC. This is described, for example, in Yu et al., Cell 2012; 149:1368-80, and in Yang et al., Bio-protocol, 2023; 12(17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO4) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO4).

In some embodiments, the modified cytosine is converted to thymine, uracil, or dihydrouracil. In any such embodiments, amplification methods may comprise uracil- and/or dihydrouracil-tolerant amplification methods, such as PCR using a uracil- and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, TET3, or a TET2 comprising a T1372S mutation. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH3CN), lithium borohydride (LiBH4), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof. Partitioning

The methods of the disclosure employ a partitioning step, wherein nucleic acids are partitioned into two or more partitions (i.e. subsamples) based on the presence or absence of 5hmC nucleic acid bases in the sample nucleic acids. The partitioning step can be performed before or after the conversion step. When the partitioning step is performed before the conversion step, one or more (e.g., both) subsamples can be carried forward to the subsequent conversion, amplification and sequencing steps. Similarly, when the partitioning step is performed after the conversion step, one or more (e.g., both) subsamples can be carried forward to the subsequent amplification and sequencing steps.

When multiple subsamples are carried forward, adapters comprising partition tags can be applied to each of the subsamples such that the subsamples can be sequenced in the same sequencing reaction while still allowing the sequencing data from each subsample to be distinguished. Tagged partitions can therefore be pooled together for collective sample prep and/or sequencing.

Partitioning can be performed using an agent which: (i) directly binds 5hmC; (ii) binds to a derivative of 5hmC; or (iii) binds to an isolation tag which has been conjugated to 5hmC.

In some embodiments, partitioning comprises exposing the nucleic acids to a binding agent which selectively binds 5hmC relative to 5mC. In some embodiments, the binding agent is an anti-5hmC antibody or an antigen binding fragment thereof. Exemplary antibodies include the antibody under catalog number 39069 from Active Motif.

In some embodiments, partitioning comprises exposing the nucleic acids to a binding agent which selectively binds to an isolation tag which has been conjugated to 5hmC. The isolation tag may be conjugated to 5hmC through chemical labeling such as through “click chemistry”. In some embodiments, the conjugation of the isolation tag comprises: (i) incubating the nucleic acids with a β-glucosyltransferase and UDP glucose modified with a chemoselective group, thereby covalently labelling the 5hmC with the chemoselective group; and (ii) linking a biotin moiety to the chemoselectively-modified 5hmC via a cycloaddition reaction. The partitioning can then be performed by binding the product of step (ii) to a support that binds to biotin (e.g., beads comprising streptavidin, such as magnetic beads comprising streptavidin). In some embodiments, the UDP glucose modified with chemoselective group is UDP-6-N3-Glu. In some embodiments, the biotin moiety is dibenzocyclooctyne-modified biotin. In some embodiments, the β-glucosyltransferase is T4 DNA-glucosyltransferase.

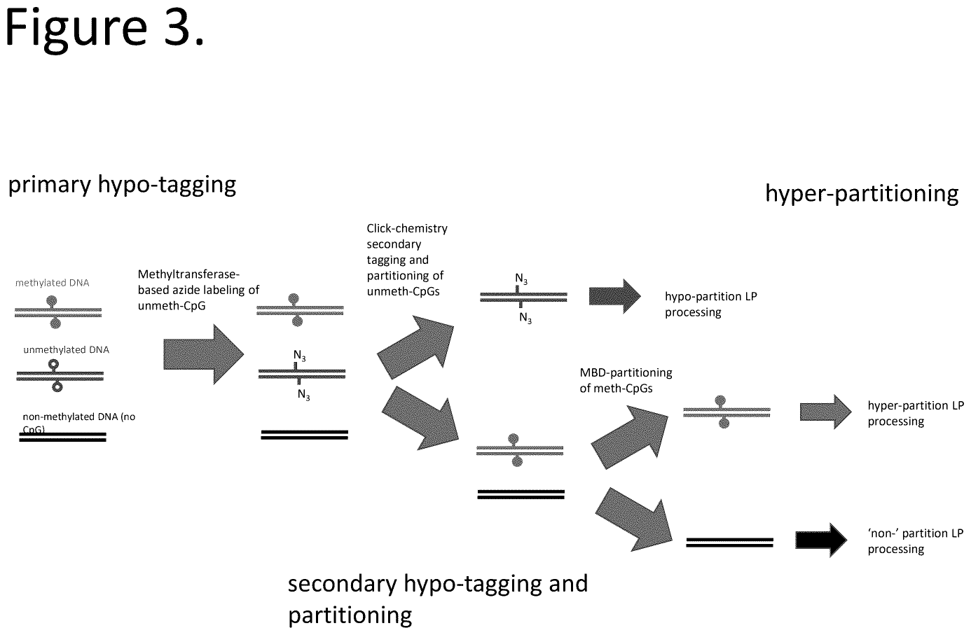

The exemplary workflow shown in FIG. 3 shows the “5hmC-SEAL” method. B-glucosyltransferase (BGT) is first applied to DNA with a UDP-6-N3-Glu substrate. This reacts selectively with 5hmC bases, resulting in a glucose moiety and N3 being transferred to the 5hmC. Standard copper-free (Cu-free) click chemistry with DBCO-biotin then is performed, in which the DBCO and N3 react, transferring biotin to the 5hmC-originating base. Streptavidin-magnetic beads can then be applied to the nucleic acid sample to isolate ('pull-down') the biotinylated-DNA, corresponding to originating nucleic acids containing 5hmC bases. Accordingly, the workflow of FIG. 3 provides at least two subsamples of nucleic acids, wherein a first subsample is enriched for nucleic acids comprising 5hmC nucleic acid bases and wherein a second subsample is depleted of nucleic acids comprising 5hmC nucleic acid bases. Such a method is described in WO 2017/176630, which is incorporated herein by reference in its entirety.

In some embodiments, partitioning comprises exposing the nucleic acids to a binding agent which selectively binds to an isolation tag which has been conjugated to 5hmC. The isolation tag may be conjugated to 5hmC through chemical labeling. The isolation tag may be a glucose residue conjugated to 5hmC DNA (i.e. the glucose residue in β-glucosylated-5hmC). In some embodiments, the conjugation of the isolation tag comprises incubating the nucleic acids with a β-glucosyltransferase and UDP-glucose, thereby covalently labelling the 5hmC with a β-glucosyl residue. The partitioning can then be performed by exposing the nucleic acids to an agent with binds glucosylated 5hmC (e.g., J-binding protein 1 (JBP1), such as biotinylated JBP1 or JBP1 bound to a support). In some embodiments, the biotinylated JBP1, and any bound nucleic acids, can then be isolated using a support (e.g., beads) comprising streptavidin. In some embodiments, the β-glucosyltransferase is T4 DNA β-glucosyltransferase. Exemplary methods are known as the JBP-1-seq method, as described in Cui et al., Genomics. 2014 p368-375, which is incorporated by reference.

After partitioning, either or both of the subsamples can be amplified and sequenced. The partition depleted in 5hmC containing nucleic acids may also be amplified and sequenced. The sequenced nucleic acids in the enriched partition can be deemed to have contained a 5hmC base at one of the cytosines present in the sequence. As 5hmC bases are relatively rare in nature, by analyzing per base coverage across sequenced nucleic acids, the location of the 5hmC bases can be estimated with high confidence.

### Amplification

Sample nucleic acids flanked by adapters can be amplified by PCR and other amplification methods. Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters when the sample nucleic acids have been subjected to A-tailing, e.g., using T4 polymerase or Klenow large fragment. This increases the efficiency of ligation and results in amplification of at least 50, 60, 70 or 80% of double stranded nucleic acids. Such methods can increase the amount or number of amplified molecules relative to control methods performed with T-tailed adapters alone by at least 10, 15 or 20%.

Amplification is performed after the conversion and partitioning steps. Amplification may be performed before or after any sequence capture step. In some embodiments, the ligating occurs before or simultaneously with amplification. In some embodiments, amplification is primed by primer binding to primer binding site(s) in the adapter(s).

### Capturing Using Capture Probes

Nucleic acids in a sample can be subject to a sequence capture step, in which molecules having target sequences are captured for subsequent analysis. Capture may be performed using any suitable approach known in the art. Target capture can involve use of a bait set comprising oligonucleotide baits labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, the methods herein comprise capturing nucleic acids comprising epigenetic and/or sequence-variable target regions. Such regions may be captured from a sample (e.g., a subsample) that has undergone attachment of adapters, conversion, partitioning, and/or amplification). Enriching for or capturing DNA comprising epigenetic and/or sequence-variable target regions may comprise contacting the DNA with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample and/or the second subsample, e.g., the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject (e.g., test subject). The target regions comprise intronic regions or VDJ regions that may comprise rearrangements, epigenetic target regions, which may show differences in methylation levels and/or fragmentation patterns depending on whether they originated from a tumor or from healthy cells, and sequence-variable regions, which may show differences in sequence, other than rearrangements, depending on whether they originated from a tumor or from healthy cells. The capturing step produces a captured set of cfDNA molecules. In some embodiments, the cfDNA molecules corresponding to the sequence-variable target region set are captured at a greater capture yield in the captured set of cfDNA molecules than cfDNA molecules corresponding to the epigenetic target region set. In some embodiments, a method described herein comprises contacting cfDNA obtained from a subject (e.g., a test subject) with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or methylation status is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, amplification is performed before the capturing step. In some embodiments, amplification is performed after the capturing step. In some embodiments, amplification is performed before and after the capturing step. In some embodiments, the methods further comprise sequencing the captured cfDNA to different degrees of sequencing depth for the epigenetic and sequence-variable target region sets and for rearrangements, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired (e.g., to partition based on methylation as described herein). These can then be pooled in appropriate proportions to provide material for further processing and analysis such as sequencing.

### Sequencing

In general, sample nucleic acids flanked by adapters can be subject to sequencing after amplification. Sequencing methods include, for example, Sanger sequencing, high-throughput sequencing, pyrosequencing, sequencing-by-synthesis, single-molecule sequencing (also known as long-read sequencing or third generation sequencing), nanopore sequencing (a type of long-read sequencing), 5-letter sequencing or 6-letter sequencing, semiconductor sequencing, sequencing-by-ligation, sequencing-by-hybridization, Digital Gene Expression (Helicos), next generation sequencing (NGS), Single Molecule Sequencing by Synthesis (SMSS) (Helicos), enzymatic methyl sequencing (EM-Seq), Tet-assisted pyridine borane sequencing (TAPS), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing unit can also include multiple sample chambers to enable processing of multiple runs simultaneously. For example, long-read sequencing (also referred to herein as single-molecule sequencing or third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing facilitates direct detection of, e.g., 5-methylcytosine and 5-hydroxymethylcytosine as well as unmodified cytosine (Weirather JL, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

ONT is a nanopore-based single molecule sequencing technology (Weirather JL, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “ID” reads (“template” and “complement”) by removing the adaptor. The consensus sequence of two “ID” reads is a “2D” read with a higher accuracy.

5-letter and 6-letter sequencing methods include whole genome sequencing methods capable of sequencing A, C, T, and G in addition to 5 mC and 5 hmC to provide a 5-letter (A, C, T, G, and either 5 mC or 5 hmC) or 6-letter (A, C, T, G, 5 mC, and 5 hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi:https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil-and/or dihydrouracil-tolerant amplification methods, such as PCR using a uracil-and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of, for example, at least 5%, 10%, 15%, 20%, 25%, 30%, 40%, 50%, 60%, 70%, or 80% of the genome. Sequence coverage can be performed on, for example, at least 5, 10, 20, 70, 100, 200 or 500 different genes, or up to, for example, 5000, 2500, 1000, 500 or 100 different genes.

Simultaneous sequencing reactions may be performed using multiplex sequencing. In some embodiments, cell-free nucleic acids may be sequenced with at least, for example, 1000, 2000, 3000, 4000, 5000, 6000, 7000, 8000, 9000, 10000, 50000, or 100,000 sequencing reactions. In other embodiments, cell-free nucleic acids may be sequenced with less than, for example, 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some embodiments, data analysis may be performed on at least, for example, 1000, 2000, 3000, 4000, 5000, 6000, 7000, 8000, 9000, 10000, 50000, or 100,000 sequencing reactions. In other embodiments, data analysis may be performed on less than, for example, 1000, 2000, 3000,4000, 5000, 6000, 7000, 8000, 9000, 10000, 50000, or 100,000 sequencing reactions. An exemplary read depth is 1000-50000 or 1000-10000 or 1000-20000 reads per locus (base).

In general, sequencing of epigenetic target regions, e.g., to analyse a methylation profile of DNA, requires a lesser depth of sequencing than sequencing of a sequence-variable target region, e.g., for analysis of mutations. Hence, lesser sequencing depths, as described herein, may in some cases be adequate for the methods described herein.

### Analyzing the Sequence Data

The sequencing data obtained by the methods of the present disclosure can be used to resolve unmethylated Cs, 5mC and 5hmC on a single molecule level.

The conversion procedures used in the methods of the present disclosure allow for 5mC to be distinguished from unmethylated Cs. For example, methods using a conversion procedure that selectively converts the base pairing specificity of 5mC means that 5mC in the sample nucleic acids will be read as T in sequencing. As noted elsewhere, 5hmC can be protected from conversion, e.g., through prior glucosylation. Aligning the sequence reads to a reference sequence (e.g., a reference genome) and identifying C>T alterations allows for the identification of 5mCs in the sample nucleic acids. Unmethylated Cs and protected 5hmCs are read as Cs in the sequencing data. The partitioning step allows 5hmCs and unmethylated Cs to be distinguished. Nucleic acids comprising a 5hmC will be partitioned into the subsample enriched for nucleic acids comprising 5hmC. Typically nucleic acids will contain at most one 5hmC nucleic acid base due to their scarcity in nature. The position of the 5hmC nucleic acid base in the nucleic acid can be identified using base coverage analysis. Base coverage analysis can involve aligning sequence reads (e.g., individual sequence reads, or consensus sequence reads for parent nucleic acids, as described elsewhere) from a subsample to a reference sequence. Analysis of the frequency (e.g., the proportion) of sequence reads which align to a specific C position in a reference sequence can identify the C position which comprised a 5hmC nucleic acid base in the sample nucleic acids. Specifically, the C position that comprised a 5hmC nucleic acid base in the sample nucleic acids would be expected to have a higher base coverage in the subsample enriched for nucleic acids comprising 5hmC nucleic acid bases compared to those C positions that did not comprise a 5hmC nucleic acid base in the sample nucleic acids.

Methods using a conversion procedure that selectively converts the base pairing specificity of unmethylated C (e.g., bisulfite sequencing) means that unmethylated C in the sample nucleic acids will be read as T in sequencing. Aligning the sequence reads to a reference sequence (e.g., a reference genome) and identifying C>T alterations allows for the identification of unmethylated Cs in the sample nucleic acids. 5mCs and 5hmCs are read as Cs in the sequencing data. The partitioning step allows 5hmCs and 5mCs to be distinguished. Nucleic acids comprising a 5hmC will be partitioned into the subsample enriched for nucleic acids comprising 5 hmC. Typically nucleic acids will contain at most one 5hmC nucleic acid base due to their scarcity in nature. The position of the 5hmC nucleic acid base in the nucleic acid can be identified using base coverage analysis, as described above.

As noted above, identifying nucleic acid bases that have undergone conversion generally involves comparing the sequence data obtained from the nucleic acids that has been subjected to the conversion procedure to a reference sequence (e.g., a reference genome). Typically, the method involves (i) comparing the sequence data with (A) one or more pre-determined reference sequence, such as reference sequences corresponding to one or more epigenetic target regions where particular significance is attached to the methylation profile, e.g., in diagnosing, prognosing or characterizing a cancer; or (B) sequence data obtained by sequencing a subsample of the nucleic acid that was not subjected to the conversion procedure, for example a subsample that was separated before subjecting a separate subsample to the conversion procedure; and (ii) identifying point differences between the converted nucleic acid sequences and the reference sequence(s) (A) or non-converted nucleic acid sequences (B) as nucleosides (in the initial sample) having a methylation status that permits a change in base pairing specificity on exposure to the conversion procedure.

The identification of the methylation status of the sample nucleic acids has a variety of utilities. For example, methylation status can be used to characterize disease states, including for example, identifying the presence or absence of cancer, identification of cancer type, and/or identifying the tissue of origin of cfDNA molecules.

Analyzing the sequence data may also include the analysis of non-methylation features, such as fragmentation patterns (e.g., in the case of cfDNA analysis) or genetic variants (such as SNVs, indels and/or CNVs). When analyzing fragmentation patterns and/or genetic variants, conversion procedure that selectively converts the base pairing specificity of 5mC are preferred. These conversion procedures are generally less destructive and thus maintain the fragmentation pattern of the sample nucleic acids. Moreover, they do not convert the base pairing specificity of unmethylated C, thus allowing for more sensitive mutation detection.

Fragmentation patterns of DNA molecules in cfDNA samples carry information about the chromatin organization of the cells or tissues from which the cfDNA fragments originate. In particular, DNA fragments released to the bloodstream is often fragmented or cleaved around nucleosomes and/or other DNA bound proteins in the cells or tissues of origin. Further, nucleosome positioning and the location of DNA binding proteins is highly tissue specific and thus is used herein to amplify signal coming from the cells or tissues from which the cfDNA fragments originate (e.g., tumor cells as well as cells in the tumor microenvironment and cells involved in the immune response). Accordingly, in some embodiments, analyzing the sequencing data may comprise analyzing the methylation profile and the fragmentation pattern of cfDNA. Such analysis can be used to identify the tissue of origin of the cfDNA and/or diagnose or prognose cancer. In some embodiments, analyzing the sequencing data may comprise analyzing the methylation profile and the presence or absence of genetic variants in cfDNA. Such analysis can be used to identify the tissue of origin of the cfDNA and/or diagnose or prognose cancer.

In some embodiments, analyzing the sequencing data may comprise analyzing: (i) the methylation profile; (ii) the fragmentation pattern; and (iii) the presence or absence of genetic variants in cfDNA. Such analysis can be used to identify the tissue of origin of the cfDNA and/or diagnose or prognose cancer.

### Modification Sensitive Sequencing

In some embodiments, the nucleic acids are linked at both ends to Y-shaped adapters including primer binding sites and tags. The molecules are amplified The methods disclosed herein may use modification sensitive sequencing to detect the modification status of one or more nucleotides. This may include nucleotides present in the original sample and/or at least one type of dNTP comprising a modified base (such as mCTP) used in the end repair reaction. In some embodiments, a DNA sample comprising a plurality of DNA molecules is subjected to modification-sensitive sequencing to obtain sequencing data derived from the DNA sample, wherein the modification-sensitive sequencing comprises subjecting the plurality of DNA molecules to a procedure that affects a first nucleobase of the plurality of DNA molecules differently from a second nucleobase of the plurality of DNA molecules, wherein the first nucleobase is a modified or unmodified nucleobase, the second nucleobase is a modified or unmodified nucleobase different from the first nucleobase, and the first nucleobase and the second nucleobase have the same base pairing specificity, thereby producing a plurality of converted DNA molecules comprising one or more inappropriately converted bases and/or one or more inappropriately unconverted bases at one or more locations. Such embodiments may also comprise a step of end-repair prior to the modification-sensitive sequencing.

Modification sensitive sequencing involves a sequencing workflow which is capable of distinguishing at least two modification states of a nucleotide bases. These two states may be: (i) whether a base is modified or not (e.g. 5mC and/or 5hmC vs unmethylated cytosine); or (ii) the type of modification which a base exhibits (e.g. 5mC vs 5hmC). Modification sensitive sequencing does not necessarily require that a specific type of modification is identified as present or absent at a specific position, just whether one or more modification types (e.g. 5mC and 5hmC) is present or absent. For instance, in some embodiments, modification sensitive sequencing includes sequencing comprising a bisulfite conversion step which can distinguish 5mC and 5hmC from unmethylated C, but it cannot distinguish between 5mC and 5hmC. In some embodiments, the modification-sensitive sequencing comprises subjecting a DNA sample (such as a DNA sample from a subject) to a procedure that affects a first nucleobase of the DNA differently from a second nucleobase of the DNA, wherein the first nucleobase is a modified or unmodified nucleobase, the second nucleobase is a modified or unmodified nucleobase different from the first nucleobase, and the first nucleobase and the second nucleobase have the same base pairing specificity.

The type of modification sensitive sequencing used will depend on the type of modified base(s) used in the end repair, such that the type of modification sensitive sequencing will be able to detect at least the presence or absence of at least that modified base.

As outlined below, there are various methods of detecting and/or identifying modified nucleosides that rely on a conversion procedure that changes the base-pairing specificity of a nucleoside, based on the modification status of the nucleosides. These changes of base-pairing specificity can then be detected, and thus the modification status of the nucleoside inferred, by sequencing. Together, the conversion procedure and the sequencing itself constitutes one form of modification aware sequencing, as referred to herein.

In some cases, a conversion procedure used in the methods of the disclosure is one that changes the base pairing specificity of a modified nucleoside (e.g. methylated cytosine), but does not change the base pairing specificity of the corresponding unmodified nucleoside (e.g. cytosine) or does not change the base pairing specificity of any un-modified nucleoside (e.g. cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as TAPS may in some cases be preferred over methods such as bisulfite sequencing and EM-seq because they are less destructive (especially important for low yield samples such as cfDNA or FFPE samples) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized regions of interest, these non-random (localized) non-conversion events can appear as false negatives (non-methylated regions). Random non-conversion methods can maximally affect a low percent of bases within a region, and thus the specificity of methylation change detection can be maximized (reduce false positives) by placing a threshold on percentage of bases within a region that are methylated/non-methylated. Hence, in some cases, a conversion procedure that does not involve denaturation is preferred.

In other cases, a conversion procedure used in the methods of the disclosure is one that changes the base pairing specificity of an unmodified nucleoside (e.g. cytosine), but does not change the base pairing specificity of the corresponding modified nucleoside (e.g. methylated cytosine such as 5hmC and/or 5mC). Such methods include, for example, bisulfite sequencing.

The skilled person can select a suitable method according to their needs, including which nucleoside modifications are to be detected and/or identified and which type of modified base is used in the end repair reaction.

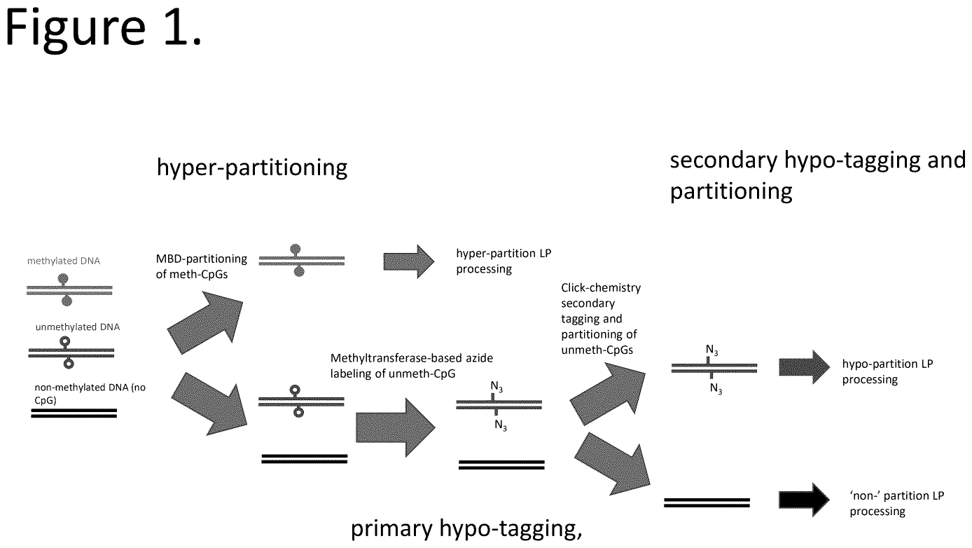

In some embodiments, the conversion procedure converts modified nucleosides. In some embodiments, the conversion procedure which converts modified nucleosides comprises Tet-assisted conversion with a substituted borane reducing agent, optionally wherein the substituted borane reducing agent is 2-picoline borane, borane pyridine, tert-butylamine borane, ammonia borane or pyridine borane. In Tet-assisted pic-borane conversion with a substituted borane reducing agent conversion, a TET protein is used to convert 5mC and 5hmC to 5caC, without affecting unmodified C. 5caC, and 5fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Sequencing of the converted DNA identifies positions that are read as cytosine as being unmodified C positions. Meanwhile, positions that are read as T are identified as being T, 5mC, 5fC, 5caC, or 5hmC. Performing TAP conversion, such as on a DNA sample as described herein, thus facilitates identifying positions containing unmodified C using the sequence reads obtained.

Hence, in these embodiments, the end repair reaction can be performed with dNTPs, wherein the at least one type of dNTP comprises a 5mC or 5hmC, and regions synthesized during the end repair reaction can be identified as those regions comprising 5mC or 5hmC (via T being called at positions which are C in the reference) at non-CpG positions. This procedure encompasses Tet-assisted pyridine borane sequencing (TAPS), described in further detail in Liu et al. 2019, supra. In this method Tet enzyme is used to progressively oxidize 5mC and 5hmC to 5fC or 5caC, then pyridine borane deaminates 5fC, 5CaC to DHU, amplified as T.

Alternatively, protection of 5hmC (e.g., using BGT or 5-hydroxymethylcytosine carbamoyltransferase) can be combined with Tet-assisted conversion with a substituted borane reducing agent, e.g. as described above. In this method (TAPS-β), 5hmC can be protected from conversion, for example through glucosylation using β-glucosyl transferase (BGT), forming (forming 5-glucosylhydroxymethylcytosine) 5ghmC, or through carbamoylation using 5- hydroxymethylcytosine carbamoyltransferase, forming 5cmC. This is described in Yu et al., Cell 2012; 149:1368-80. Treatment with a TET protein such as mTet 1 then converts 5mC to 5caC but does not convert C, 5ghmC, or 5cmC. 5 caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either 5hmC or unmodified C positions. Meanwhile, positions that are read as T are identified as being T, 5fC, 5caC, or 5mC. Performing TAPSβ conversion on a sample as described herein thus facilitates distinguishing positions containing unmodified C or 5hmC on the one hand from positions containing 5mC using the sequence reads obtained. Hence, in these embodiments, the end repair reaction can be performed with dNTPs, wherein the at least one type of dNTP comprises a 5mC, and regions synthesized during the end repair reaction can be identified as those regions comprising 5mC (via T being called at positions which are C in the reference) at non-CpG positions. For an exemplary description of this type of conversion, see, e.g., Liu et al., Nature Biotechnology 2019; 37:424-429. 5- hydroxymethylcytosine carbamoyltransferase is described in Yang et al., Bio-protocol, 2023; 12(17): e 4496.

In some embodiments, the conversion procedure converts modified nucleosides. In some embodiments, the conversion procedure which converts modified nucleosides comprises chemical-assisted conversion with a substituted borane reducing agent, optionally wherein the substituted borane reducing agent is 2-picoline borane, borane pyridine, tert-butylamine borane, borane pyridine or ammonia borane. In chemical-assisted conversion with a substituted borane reducing agent, an oxidizing agent such as potassium perruthenate (KRuO4) (also suitable for use in ox-BS conversion) is used to specifically oxidize 5 hmC to 5fC. Treatment with pic-borane or another substituted borane reducing agent such as borane pyridine, tert-butylamine borane, or ammonia borane converts 5 fC and 5 caC to DHU but does not affect 5mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either 5mC or unmodified C positions. Meanwhile, positions that are read as T are identified as being T, 5fC, 5caC, or 5hmC. Performing this type of conversion as described herein thus facilitates distinguishing positions containing unmodified C or 5mC on the one hand from positions containing 5hmC using the sequence reads obtained. Hence, in these embodiments, the end repair reaction can be performed with dNTPs, wherein at least one type of dNTP comprises a 5hmC, and regions synthesized during the end repair reaction can be identified as those regions comprising 5hmC (via T being called at positions which are C in the reference) at non-CpG positions. For an exemplary description of this type of conversion, see, e.g., Liu et al., Nature Biotechnology 2019; 37:424-429.

Exemplary conversion procedures that change the base-pairing specificity of modified cytosines have been described. However, the methods described herein could in principle use any modified nucleoside and suitable conversion procedure (i.e. single-base epigenetic conversion assay) that changes the base-pairing specificity of the modified nucleoside and thereby allows the modified base to be distinguished from the corresponding unmodified nucleoside and/or other types of modification when sequenced. For example, any conversion procedure could be used allowing any one of N6-methyladenine (6mA), N6-hydroxymethyladenine (6hmA), or N6-formyladenine (6fA) to be distinguished from unmodified adenosine.

In some embodiments, the conversion procedure converts unmodified nucleosides. In some embodiments, the conversion procedure which converts unmodified nucleosides comprises bisulfite conversion. Treatment with bisulfite converts unmodified cytosine and certain modified cytosine nucleotides (e.g. 5-formyl cytosine (5fC) or 5- carboxylcytosine (5ca C)) to uracil whereas other modified cytosines (e.g., 5mC and 5hmC) are not converted. Thus, where bisulfite conversion is used, the first nucleobase comprises one or more of unmodified cytosine, 5fC, 5caC, or other cytosine forms affected by bisulfite, and the second nucleobase may comprise one or more of 5mC and 5hmC, such as 5mC and optionally 5hmC. Sequencing of bisulfite-treated DNA identifies positions that are read as cytosine as being 5mC or 5hmC positions. Meanwhile, positions that are read as T are identified as being T or a bisulfite-susceptible form of C, such as unmodified cytosine, 5fC, or 5caC. Thus, performing bisulfite conversion, such as on a DNA sample as described herein facilitates identifying positions containing 5mC or 5hmC. Hence, in these embodiments, the end repair reaction can be performed with dNTPs, wherein at least one type of dNTP comprises a 5mC and/or a 5hmC, and regions synthesized during the end repair reaction can be identified as those regions comprising 5mC or a 5hmC (via C being called at these positions) at non-CpG positions. For an exemplary description of bisulfite conversion, see, e.g., Moss et al., Nat Commun. 2018; 9:5068.

In some embodiments, the procedure which converts unmodified nucleosides comprises oxidative bisulfite (Ox-BS) conversion. This procedure first converts 5hmC to 5fC, which is bisulfite susceptible, followed by bisulfite conversion. Thus, when oxidative bisulfite conversion is used, the first nucleobase comprises one or more of unmodified cytosine, 5fC, 5caC, 5hmC, or other cytosine forms affected by bisulfite, and the second nucleobase comprises 5mC. Sequencing of Ox-BS converted DNA identifies positions that are read as cytosine as being 5mC positions. Meanwhile, positions that are read as T are identified as being T or a bisulfite-susceptible form of C, such as unmodified cytosine, 5fC, or 5hmC. Hence, in these embodiments, the end repair reaction can be performed with dNTPs, wherein at least one type of dNTP comprises a 5mC, and regions synthesized during the end repair reaction can be identified as those regions comprising 5mC (via C being called at these positions) at non-CpG positions. Performing Ox-BS conversion thus facilitates identifying positions containing mC. For an exemplary description of oxidative bisulfite conversion, see, e.g., Booth et al., Science 2012; 336:934-937.

In some embodiments, the procedure which converts unmodified nucleosides comprises Tet-assisted bisulfite (TAB) conversion. In TAB conversion, 5hmC is protected from conversion and 5mC is oxidized in advance of bisulfite treatment, so that positions originally occupied by 5mC are converted to U while positions originally occupied by 5hmC remain as a protected form of cytosine. For example, as described in Yu et al., Cell 2012; 149:1368-80, β-glucosyl transferase can be used to protect 5hmC (forming 5-glucosylhydroxymethylcytosine (5ghmC)), then a TET protein such as mTet 1 can be used to convert 5mC to 5caC, and then bisulfite treatment can be used to convert C and 5caC to U while 5ghmC remains unaffected.

Alternatively, a carbamoyltransferase enzyme, such as 5-hydroxymethylcytosine carbamoyltransferase as described in Yang et al., Bio-protocol, 2023; 12(17): e4496, can be used to protect hmC (by converting hmC to 5-carbamoyloxymethylcytosine (5cmC)), then a TET protein such as mTet1 can be used to convert mC to caC, and then bisulfite treatment can be used to convert C and caC to U while 5cmC remains unaffected. Thus, when TAB conversion is used, the first nucleobase comprises one or more of unmodified cytosine, 5fC, 5caC, 5mC, or other cytosine forms affected by bisulfite, and the second nucleobase comprises 5hmC. Sequencing of TAB-converted DNA identifies positions that are read as cytosine as being 5hmC positions.

Meanwhile, positions that are read as T are identified as being T, or a bisulfite-susceptible form of C, such as unmodified cytosine, 5mC, 5fC, or 5caC. Performing TAB conversion on a first subsample as described herein thus facilitates identifying positions containing 5hmC. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions.

In some embodiments, the conversion procedure which converts unmodified nucleosides comprises APOBEC-coupled epigenetic (ACE) conversion. In ACE conversion, an AID/APOBEC family DNA deaminase enzyme such as APOBEC3A (A3A) is used to deaminate unmodified cytosine and 5mC without deaminating 5hmC, 5fC, or 5caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being 5hmC, 5fC, or 5caC positions. Meanwhile, positions that are read as T are identified as being T, unmodified C, or 5mC. Performing ACE conversion as described herein thus facilitates distinguishing positions containing 5hmC from positions containing 5mC or unmodified C using the sequence reads obtained from the first subsample. Hence, in these embodiments, the end repair reaction can be performed with dNTPs, wherein at least one type of dNTP comprises a 5hmC, and regions synthesized during the end repair reaction can be identified as those regions comprising 5hmC (via C being called at these positions) at non-CpG positions. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12(17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36:1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M. SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.Mpel). In a particular embodiment, the methyltransferase enzyme is a variant of M.Mpel, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.Mpel having an N374R substitution or an N374K substitution. The methyltransferase can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5hmCs, such as by glucosylation of the 5hmCs (e.g., using BGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (βGT), forming (5-glucosylhydroxymethylcytosine) 5ghmC, or through carbamoylation using 5- hydroxymethylcytosine carbamoyltransferase, forming 5cmC. This is described, for example, in Yu et al., Cell 2012; 149:1368-80, and in Yang et al., Bio-protocol, 2023; 12(17): e4496.

Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A).

Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO4) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO4).

In some embodiments, the modified cytosine is converted to thymine, uracil, or dihydrouracil. In any such embodiments, amplification methods may comprise uracil-and/or dihydrouracil-tolerant amplification methods, such as PCR using a uracil-and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH3CN), lithium borohydride (LiBH4), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof. Various TET enzymes may be used in the disclosed methods as appropriate, as described elsewhere herein.

Modification sensitive sequencing also includes sequencing methods which do not rely on a conversion step, wherein the base pairing specificity of a base is changed dependent on its modification status. For instance, single molecule techniques such as nanopore based sequencing and single molecule real time sequencing can be used to directly detect modified bases.

For example, some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore. One example of a nanopore sequencing system is that commercialized by Oxford Nanopore Technologies (ONT). (See e.g., (Weirather et al., F1000Research, 6:100, 2017.) ONT sequencing directly sequences a native single-stranded DNA (ssDNA) molecule by measuring characteristic current changes as the bases are threaded through the nanopore by a molecular motor protein. ONT sequencing uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “1D” reads (“template” and “complement”) by removing the adaptor. The consensus sequence of two “1D” reads is a “2D” read with a higher accuracy.

Nanopore sequencing can be used to detect base modifications including 5mC, 5hmC, 6mA, BrdU, FdU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the modification sensitive sequencing comprises nanopore sequencing. In such embodiments, the end repair may be performed using dNTPs, which comprise 4mC, 5mC, 5hmC, 6mA, BrdU, FdU, IdU, and/or EdU.

Another modification sensitive single molecule sequencing technique is single molecule real time sequencing (SMRT) that has been commercialized by Pacific Biosciences. SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.) Single molecule real time sequencing can be used to detect base modifications such as 4mC, 5 mC, 5hmC, 6mA, and 8ox0G (Gouil & Keniry Essays in Biochemistry (2019) 63 639-648). Accordingly, in some embodiments, the modification sensitive sequencing comprises single molecule real time sequencing. In such embodiments, the end repair may be performed using dNTPs, which comprise 4mC, 5 mC, 5 hmC, 6mA, and/or 80x0G.

### Partitioning

In some instances, a heterogeneous nucleic acid sample is partitioned into two or more partitions (sub-samples). In some embodiments, each partition is differentially tagged. Tagged partitions can then be pooled together for collective sample prep and/or sequencing. The partitioning-tagging-pooling steps can occur more than once, with each round of partitioning occurring based on a different characteristics, and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more base modifications and without the one or more base modifications. Examples of base modifications are described elsewhere herein. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some cases, different procedures are applied to different partitions to determine different characteristics of the initial sample. The DNA of at least one partition is subjected to an end repair and modification sensitive sequencing procedure according to the methods of the disclosure described herein. In some embodiments at least one partition is not subjected to the end repair and modification sensitive sequencing procedure according to the methods of the disclosure described herein. In cases where the modification sensitive sequencing procedure comprises a conversion procedure, corresponding sequences from the converted and non-converted partitions can be compared to identify single nucleotides that have undergone conversion and therefore identify corresponding modified nucleosides in the initial sample.

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to a molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Disclosed methods herein comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation).

In some embodiments, methylation levels can be determined using partitioning, modification-sensitive conversion such as bisulfite conversion, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner@Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, WO2024/159053, WO2021/236778, each of which are incorporated herein by reference.

In some embodiments, the nucleic acid molecules can be partitioned into different partitions based on the nucleic acid molecules that are bound to a specific protein or a fragment thereof and those that are not bound to that specific protein or fragment thereof.

Nucleic acid molecules can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acid molecules based on protein bound regions. Examples of methods used to partition nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with capture probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

### EXAMPLES

### Example 1

In one example of a workflow, the sample nucleic acids are first subjected to 5hmC glucosylation (e.g., with βGT), followed by biotinylation as described in detail elsewhere. The nucleic acids comprising biotin-labelled 5hmC are then enriched using magnetic beads comprising streptavidin. One or more of the resulting partitions are then subjected to a conversion procedure that selectively converts the base pairing specificity of 5-methylcytosines (5mC) such as TAPS or DM-Seq. The 5hmC nucleic acid bases are not converted due to them being protected through the prior modifications. After subsequent amplification and sequencing, 5mC nucleic acid bases can be identified through C>T alterations relative to a reference sequence. 5hmC nucleic acid bases can be identified through the presence of cytosines with a higher read coverage in the subsample that is enriched for nucleic acids comprising 5hmC nucleic acid bases.

Then, the sample nucleic acids are first subjected to 5hmC glucosylation (e.g., with GT), followed by a conversion procedure that selectively converts the base pairing specificity of 5-methylcytosines (5mC) such as TAPS or DM-Seq. The 5hmC nucleic acid bases are not converted due to them being protected through the prior glucosylation. The glucosylated 5hmCs then undergo biotinylation as described in detail elsewhere. The nucleic acids comprising biotin-labelled 5hmC are then enriched using magnetic beads comprising streptavidin. One or more of the resulting partitions are then subjected to amplification and sequencing. The 5mC nucleic acid bases can be identified through C>T alterations relative to a reference sequence. 5hmC nucleic acid bases can be identified through the presence of cytosines with: (i) a higher read coverage in the subsample that is enriched for nucleic acids comprising 5hmC nucleic acid bases; and/or (ii) the lower read coverage in the subsample that is depleted for nucleic acids comprising 5hmC nucleic acid bases.

### Example 2

As described, DM-seq is an enzymatic method that selectively converts 5mC, to be read as T in sequencing. Accordingly, when analyzing the sequence data, C>T variants are identified as 5mC in the sample nucleic acids. The method involves applying a DNA βGT to nucleic acids which selectively glycosylates 5hmC to 5ghmC. Subsequently a methyltransferase variant with carboxy-methyltransferase activity (CxMTase) is applied to the nucleic acids, selectively carboxylating the cytosine bases in the DNA to 5-carboxymethylcytosine (5cxmC). In some embodiments, the βGT and CxMTase reactions are applied simultaneously. After CxMTase treatment, APOBEC3A is applied to the nucleic acids which selectively deaminates 5mC. Upon DNA amplification and sequencing, both C-and 5hmC-originating bases will be amplified/read as C, while 5mC-originating bases will be amplified and read as T. 5 mC bases can be identified in analysis by identifying C>T mutation with respect to a reference genome.

The described workflow is compatible with Tet-assisted pyridimidine borane sequencing ‘beta’ (TAPS-β) conversion procedure. TAPS-β is an enzymatic and chemical method to selectively convert 5mC, which is read as T in sequencing (C>T variant are identified as 5mC). The method involves applying a beta-glucosyltransferase (BGT) to DNA molecules which selectively glycosylates 5hmC to 5ghmC. Subsequent application of TET enzyme selectively and sequentially oxidates 5mC bases to higher oxidation cytosine states, 5-formyl-methylcytosine and 5-carboxylcytosine. Both of these states are substrates for deamination to dihydrouracil (DHU) by borane compounds (e.g., pyrimidine borane). Upon amplification, the DHU bases are converted to T. In sequencing, sequencing, both C-and 5hmC-originating bases will be read as C, while 5mC-originating bases will be amplified and read as T. 5 mC bases can therefore be identified in analysis by identifying C>T mutation with respect to a reference sequence.

FIG. 3 shows a method involving partitioning which can be used in the methods of the disclosure. This method is known as 5hmC-SEAL. βGT is first applied to DNA with a UDP- 6-N3-Glu substrate. This reacts selectively with 5hmC bases, resulting in a glucose moiety and N3 being transferred. Standard copper-free click chemistry with DBCO-biotin then is performed, in which the DBCO and N3 react, transferring the biotin to the 5hmC-originating base. Streptavidin-magnetic beads are then applied to the DNA to isolate the biotinylated-DNA, corresponding to originating molecules containing 5hmC bases. After magnetic bead DNA isolation, the partition enriched for nucleic acids comprising the 5hmC can be amplified and sequenced. These nucleic acids will typically have a 5hmC base at one of the cytosines. As 5hmC bases are relatively rare in the human genome, by analyzing per base coverage across sequenced fragments the location of the 5hmC bases can be estimated with high confidence.

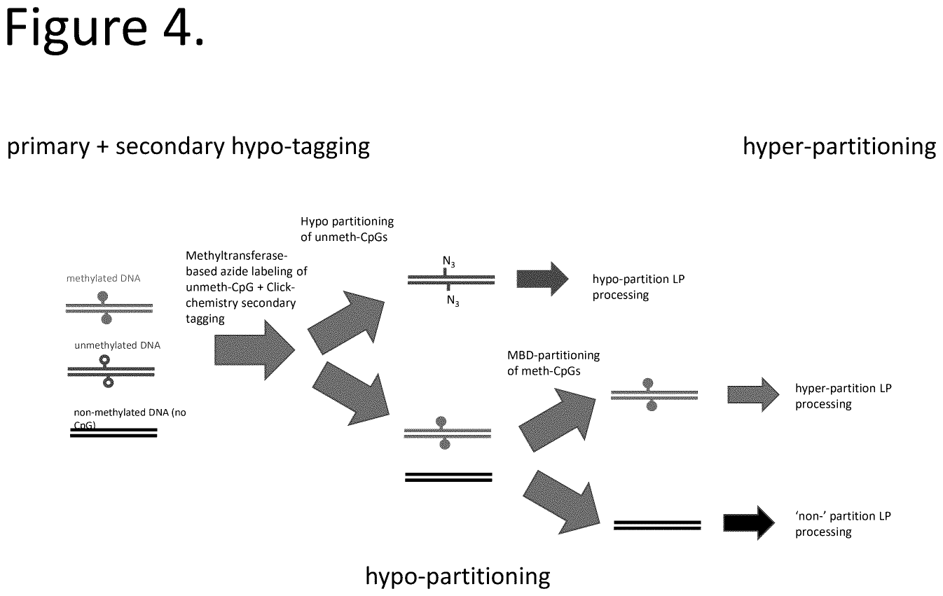

FIG. 4 schematically represents embodiments of the present disclosure in which 5hmC glucosylation and biotinylation are followed by a conversion step, which is then followed by the partitioning step.

### Example 3

Further described is an exemplary DM-Seq-involved 5mC, 5hmC resolved workflow. βGT is first applied to DNA with a UDP- 6-N3-Glu substrate. This reacts selectively with 5hmC bases, resulting in a glucose moiety and N3 being transferred. Standard copper-free click chemistry with DBCO-biotin then is performed, in which the DBCO and N3 react, transferring biotin to the 5hmC-originating base. Subsequently a methyltransferase variant with carboxy-methyltransferase activity (CxMTase) is applied to the DNA, selectively carboxylating the cytosine bases in the DNA to 5-carboxymethylcytosine (5cxmC). The βGT and CxMTase reactions may be applied simultaneously. After CxMTase treatment, APOBEC3A is applied to the DNA and will selectively deaminate 5mC. Streptavidin-magnetic beads are then applied to the DNA to partition the biotinylated-DNA, corresponding to originating molecules containing 5hmC bases. The DNA is then amplified, in which both C-and 5hmC-originating bases will be amplified as C, while 5mC-originating bases will be amplified as T. Upon sequencing, 5mC bases can be identified in analysis by identifying C>T mutation with respect to a reference sequence. Sequenced molecules in the 5hmC-enriched partition will typically contain a 5hmC base at one of the cytosines present in the read. As 5hmC bases are relatively rare in nature, by analyzing per base coverage across sequenced fragments the location of the 5hmC bases can be estimated with high confidence. Lower coverage bases read as cytosine in the 5hmC-enriched partition, and all read-as-cytosine bases in non-5hmC enriched partition are identified as unmethylated Cs in the sample nucleic acids. Thus, C, 5mC and 5hmC bases are identified and resolved in a single workflow.

An exemplary TAPS-β involved 5mC, 5hmC resolved workflow is described. βGT is first applied to DNA with a UDP-6-N3-Glu substrate. This reacts selectively with 5hmC bases, resulting in a glucose moiety and N3 being transferred. Standard copper-free click chemistry with DBCO-biotin then is performed, in which the DBCO and N3 react, transferring biotin to the 5hmC-originating base. Subsequent application of TET enzyme selectively and sequentially oxidates 5mC bases to higher oxidation cytosine states—5-formyl-methylcytosine and 5-carboxylcytosine. Both of these states are substrates for deamination to dihydrouracil (DHU) by borane compounds (e.g., pyrimidine borane) in the next step. Streptavidin-magnetic beads are then applied to the DNA to partition the biotinylated-DNA, corresponding to originating molecules containing 5hmC bases. Upon amplification, the DHU bases are converted to T. In sequencing, both C- and 5 hmC-originating bases will be read as C, while 5mC-originating bases will be amplified and read as T. 5mC bases can be identified in analysis by identifying C>T mutation with respect to a reference sequence. Sequenced molecules in the 5hmC-enriched partition will typically contain a 5hmC base at one of the cytosines present in the read. As 5hmC bases are relatively rare in the human genome, by analyzing per base coverage across sequenced fragments the location of the 5hmC bases can be estimated with high confidence. Lower coverage bases read as cytosine in the 5hmC-enriched partition, and all read-as-cytosine bases in non-5hmC enriched partition are identified as unmethylated Cs in the sample nucleic acids. Thus, C, 5mC and 5hmC bases are identified and resolved in a single workflow.

In various workflows the partitioning step occurs before the conversion procedure. In these workflows, one or more (e.g., both) subsamples obtained from the partitioning step can be carried forward into the conversion, amplification and sequencing steps. As the CxMTase reaction acts on double stranded DNA (not single stranded DNA), the conversion procedure must be performed before elution from the magnetic beads. Similarly, as the TET oxidation reaction acts preferentially on double stranded DNA (relative to single stranded DNA), it may be advantageous to perform this conversion step before elution from the magnetic beads.

Direct base analysis of C>T changes with respect to a reference sequence can be used to identify the unmethylated cytosine bases. Base coverage analysis of the 5hmC-enriched partition can then be applied to identify which of the read cytosines originated as 5hmC, and the remaining read cytosines are determined to be 5mC.

### Example 4

As described, DM-seq involves use of two enzymes:
1. MTase-based tagging of DNA (modifies unmethylated cytosines in CpGs with azide-terminated alkyne)
2. Cytosine deaminase reaction (e.g. APOBEC3A)-deaminates methylated cytosines in CpG context and all cytosines in CpH contest
+library prep and sequencing or qPCR readout of methylation (observed TpG=methylated CpG, observed CpG=unmethylated CpG)

### Example 5

In an exemplary illustrative protocol:
1. methyltransferase (MTase) MTase-based tagging of DNA (modifies unmethylated cytosines in CpGs with azide-terminated alkyne)
2. End-repair, A-tailing (optional), adapter ligation (cytosine in adapter protected from deaminase conversion)
3. Cytosine deaminase reaction (e.g. APOBEC3A)—deaminates methylated cytosines in CpG context and all cytosines in CpH contest
4. Click-chemistry reaction with immobilization affinity pair (DBCO-biotin) conjugation to azide-terminated cytosines in CpG
5. Immobilization/pull down of biotinylated DNA (contained unmethylated CpG) with streptavidin magnetic beads
6. Amplification of enriched library off of bead complexes
7. Optional hybrid capture enrichment of selected genomic regions to assay
8. Sequencing
9. Primary and secondary NGS analysis, including methylation analysis—if CpG in genome is observed in read as TpG then molecule contained methylated CpG, if read as CpG then molecule had unmethylated CpG.

### Example 6

One of ordinary skill readily appreciates that the order of steps in exemplary illustrative protocol above may be altered. An important feature is that deamination reaction occurs after original MTase tagging and before amplification. Different library-preps (.e.g ssDNA prep) can also be used and performed after deamination.

### Example 7

Enrichment-based and bisulfite-free approach for epigenomic profiling named “Active-Seq” (Azide Click Tagging for In Vitro Epigenomic sequencing) support hypomethylation analysis, including genome-wide profiling of DNA, by enriching for nonmodified CpG sites using a mutated methyltransferase enzyme. See Tosi, et al. (2023), incorporated by reference herein.

The aforementioned method can therefore be integrated in a variety of ordered partition steps, including hyper-, hypo-, non-methylated partitioning schemes, such as: (1): hyper-partitioning, primary hypo-tagging, secondary hypo-tagging and partitioning; (2): primary hypo-tagging, hyper-partitioning, secondary hypo-tagging and partitioning; (3): primary hypo-tagging, secondary hypo-tagging and partitioning, hyper-partitioning; (4): primary+secondary hypo-tagging, hypo-partitioning, hyper-partitioning; (5): primary+secondary hypo-tagging, hypo-partitioning, hyper-partitioning.

### Example 8

The described methods and compositions provide advantages by streamlining and effectuating a high efficiency method to interrogate unmethylated DNA regions with high sensitivity and low costs. Can be integrated into larger ‘integrated partitioning workflow’ to assess hyper, hypo-methylation and somatic alterations with high accuracy/sensitivity.

### Example 9

Based on the described workflows, hypomethylation detection has implications to improve cell/tissue-of-origin estimates and gene-expression inference that have clinical utility in Oncology and Cancer/non-Cancer Screening applications.

1. A method for detecting the methylation profile of nucleic acids in a sample, wherein the method comprises:
partitioning the nucleic acids based on the presence or absence of 5-hydroxymethylcytosine (5hmC) nucleic acid bases in the nucleic acids;
subjecting the nucleic acids to a conversion procedure that selectively converts the base pairing specificity of 5-methylcytosines (5mC) or unmethylated cytosines (C) in the nucleic acids;
amplifying the nucleic acids which to generate amplification products;
sequencing the amplification products to obtain sequencing data; and
analysing the sequencing data to determine whether the cytosine nucleic acid bases of the nucleic acids in the sample are 5hmC, 5mC or C.

2. The method of claim 1, wherein partitioning is performed before the conversion procedure.

3. The method of claim 1, wherein conversion is performed before partitioning.

4. The method of claim 2, wherein the partitioning provides at least two subsamples of nucleic acids, wherein a first subsample is enriched for nucleic acids comprising 5hmC nucleic acid bases and wherein a second subsample is depleted of nucleic acids comprising 5hmC nucleic acid bases, wherein amplifying, sequencing and/or analyzing are performed on at least the first subsample and/or at least the second subsample.

5. The method of claim 1, wherein the partitioning comprises modifying the 5hmC nucleic acid base by attaching an isolation tag and partitioning using an agent which binds to the isolation tag.

6. The method of claim 1, wherein the method further comprises, prior to partitioning and/or the conversion procedure, incubating the nucleic acids with β-glucosyltransferase and a uridine diphosphoglucose (UDP-Glu) molecule to glycosylate 5hmC nucleic acid bases in the nucleic acid molecule with a glucose molecule.

7. The method of claim 1, wherein partitioning comprises generation of one or more of:
a primary, secondary, or untagged hyper-partition,
a primary, secondary, or untagged hypo-partition, and
a primary, secondary, or untagged other partition.

8. The method of claim 1, wherein the partitioning comprises generation of a hyper-partition, a hypo-partition, and/or other partition.

9. The method of claim 1, the partitioning comprises generation of a primary tagged hyper-partition, a hypo-partition, and/or other partition.

10. The method of claim 1, the partitioning comprises generation of a secondary tagged a hyper-partition, a hypo-partition, and/or other partition.

11-15. (canceled)

16. The method of claim 6, wherein the modified UDP-Glu comprises an azide linker and/or a thiol linker.

17. The method of claim 16, wherein the modified UDP-Glu comprises an isolation tag which is used in the partitioning step.

18. The method of claim 17, wherein the isolation tag is biotin or a histidine tag.

19. The method of claim 6, wherein the partitioning comprises one or more of:
reacting the modified glucose with an isolation tag, and binding the glycosylated 5hmC with J binding protein 1 (JBP1).

20. (canceled)

21. The method of claim 1, wherein the partitioning comprises exposing the nucleic acids to a binding agent which selectively binds 5hmC.

22. The method of claim 21, wherein the binding agent is an anti-5hmC antibody, or an antigen-binding fragment thereof.

23. The method of claim 1, wherein the conversion procedure selectively converts the base pairing specificity of 5-methylcytosines (5mC) in the nucleic acids.

24. The method of claim 1, wherein the conversion procedure comprises Tet-assisted conversion of nucleic acids with a substituted borane reducing agent, wherein 5hmC nucleic acid bases are protected from conversion,

25. The method of claim 24, wherein the substituted borane reducing agent is 2-picoline borane, borane pyridine, tert-butylamine borane, ammonia borane or pyridine borane.

26. The method of claim 1, wherein the conversion procedure comprises:
reacting the nucleic acids with a variant methyltransferase having carboxymethyltransferase activity in the presence of carboxy-S-adenosyl-L-methionine (CxSAM) substrate, thereby labelling any unmethylated C and rendering it resistant to deaminase action, wherein 5hmC nucleic acid bases are protected from conversion through glucosylation; and
contacting the nucleic acids of step (i) with a deaminase enzyme which is APOBEC3A.

27. The method of claim 1, wherein the conversion procedure comprises unmethylated cytosine (C), 5-methylcytosine (5mC) and 5-hydroxymethylcytosine (5hmC) in nucleic acids in the sample.

28. The method of claim 1, wherein the conversion procedure, comprises:
reacting nucleic acids containing C, 5mC, and/or 5hmC with a variant methyltransferase having carboxymethyltransferase activity in the presence of carboxy-S-adenosyl-L-methionine (CxSAM) substrate, thereby labeling any unmodified C in said nucleic acids and rendering it resistant to deaminase action;
contacting the nucleic acids with a deaminase which deaminates 5mC and/or 5hmC, with minimal damage to said target polynucleotide present in said sample;
analyzing said polynucleotide sample, to identify each of unmodified C, 5mC, and 5hmC present in said polynucleotide.

29. The method of claim 1, wherein the nucleic acids in the sample are fragmented or sheared prior to partitioning and/or the conversion method.

30. The method of claim 1, wherein sequencing comprises use of sequence adapters containing modified cytosine bases resistant to deamination.

31. The method of claim 1, wherein the nucleic acids in the sample are amplified prior to sequencing.

32. The method of claim 24, wherein said variant methyltransferase having carboxymethylase activity is a recombinant M.MpeI N374K and said.

33. (canceled)

34. The method of claim 30, wherein modified cytosine base is 5pyC.

35. The method of claim 1, wherein said DNA is genomic DNA.

36. The method of claim 1, wherein said DNA is cell-free DNA (cfDNA).

37-47. (canceled)

48. The method of claim 1, further comprising using the detection of the methylation status in the nucleic acids to determine or predict the presence or absence of nucleic acids produced by a cancer cell or tumor, to determine the probability that a subject has a tumor or cancer, or to characterize a cancer or tumor of the subject.
